(19) United States
(12) Patent Application Publication
(10) Pub. No.: US 2023/0310856 A1
(43) Pub. Date: Oct. 5, 2023

(54) SYSTEMS AND METHODS FOR VAGUS NERVE STIMULATION

(71) Applicant: The Alfred E. Mann Foundation for Scientific Research, Valencia, CA (US)

(72) Inventors: Brian Michael SHELTON, Ventura, CA (US); Hitesh Hotlani, Santa Clarita, CA (US)

(21) Appl. No.: 18/194,217

(22) Filed: Mar. 31, 2023

Related U.S. Application Data
(60) Provisional application No. 63/326,228, filed on Mar. 31, 2022.

(51) Int. Cl. A61N 1/36; A61N 1/02

(52) U.S. Cl. CPC A61N 1/36053; A61N 1/36132; A61N 1/025

(57) ABSTRACT

The disclosure provides systems and methods for system for vagus nerve stimulation (VNS), using a VNS stimulator implanted in a human subject and configured to transmit electrical stimulation pulses to a vagus nerve of the human subject; and at least one sensor configured to detect a predefined pattern of tactile input, a bodily gesture, and/or a voice command from the human subject, wherein the VNS stimulator includes a controller configured to modulate at least one stimulation parameter of the electrical stimulation pulses based at least in part on the tactile input, bodily gesture, and/or voice command from the human subject.

### CROSS-REFERENCE TO RELATED APPLICATION

This application claims priority to U.S. Provisional Application No. 63/326,228, entitled “SYSTEMS AND METHODS FOR VAGUS NERVE STIMULATION,” filed Mar. 31, 2022, the entire contents of which is incorporated herein by reference in its entirety.

### BACKGROUND

Epilepsy and depression are two extremely common maladies. Epilepsy produces potentially-fatal seizures. Both conditions can be treated under appropriate circumstances with vagus nerve stimulation (VNS). VNS entails the surgical implantation of a device into a patient's chest area under the skin to stimulate the vagus nerve with electrical impulses. The vagus nerve originates from the brainstem and traverses both sides of the neck down to the chest and abdomen. The VNS device sends electrical signals via the vagus nerve to the brain. A lead wire connects the device to the vagus nerve via a cuff with one or more electrodes. VNS has been shown to be helpful in many cases for reducing the number and severity of seizures, particularly for patients who are less responsive to more non-invasive methods like oral medication. VNS has also been shown to reduce depression in certain treatment-resistant patients.

Some current VNS systems allow a patient (or clinician) to trigger a change in the stimulation parameters by swiping a magnetic controller over the implantable pulse generator (IPG), e.g., to provide a temporary stimulation boost. The purpose for this is that some patients can sense that a seizure is about to happen before the seizure starts. Triggering a boost with a higher amplitude or frequency, or a longer pulse width, can either prevent a seizure or reduce the length of the seizure. However, the patient needs to keep the magnet with them, find the magnet, and swipe it over the IPG in order to execute the manual boost, often within a brief window of time. As such, current systems are non-ideal in that they require that a patient must keep the magnetic controller on hand in case the need to trigger a boost arises, potentially resulting in anxiety and/or limited utilization of this manual control feature, and a reduction in the quality of patient care.

### BRIEF SUMMARY OF EXEMPLARY ASPECTS OF THE DISCLOSURE

The systems and methods for VNS described herein address various shortcomings in the art, e.g., by allowing users to manually adjust stimulation parameters when needed using a predefined pattern of tactile input and/or voice commands. The predefined pattern of tactile input may include one or more taps and/or bodily gestures that are sensed by a motion sensor. In a preferred embodiment, the motion sensor is in or on an implanted stimulator. In practice, such systems can provide a fast and accurate means for controlling VNS, resulting in an improved quality of life for patients that require VNS systems.

In a first general aspect, the disclosure provides a system for VNS, comprising: a VNS stimulator implanted in a human subject and configured to transmit electrical stimulation pulses to a vagus nerve of the human subject; and at least one sensor configured to detect a predefined pattern of tactile input and/or a voice command from the human subject, wherein the VNS stimulator comprises a controller configured to modulate at least one stimulation parameter of the electrical stimulation pulses based at least in part on the tactile input and/or the voice command from the human subject. In a preferred embodiment, the at least one sensor is in, on, or in proximity to the VNS stimulator.

In some aspects, the at least one stimulation parameter comprises a pulse amplitude, pulse width, duty cycle, pulse frequency, ramp-on rate, and/or ramp-off rate of the electrical stimulation pulses.

In some aspects, the controller is configured to modulate the at least one stimulation parameter in response to a predefined pattern of tactile input from the human subject. In some aspects, the controller may be configured to recognize and/or respond to multiple predefined patterns of tactile input and/or voice commands, which may be configured, e.g., to trigger different modulation responses. For example, the controller may be configured to modulate the pulse width of the electrical stimulation pulses based on or in response to a double-tap, and to modulate the pulse frequency of the electrical stimulation pulses based on or in response to a triple-tap. It is understood that any tactile input, bodily gesture, or voice command described herein may be used as a predefined pattern to trigger modulation of any of the stimulation parameters described herein.

In some aspects, the sensor comprises a motion sensor and the tactile input comprises a double-tap detected by the motion sensor. In some aspects, the sensor comprises a motion sensor and the tactile input comprises a triple-tap detected by the motion sensor. In other aspects, the tactile input may comprise additional taps (e.g., 4, 5, 6, 7, 8, 9 or 10 taps). The controller may be configured to recognize evenly-spaced taps and/or taps separated by uneven spacing (e.g. a triple-tap, wherein the taps are separated by a pause of a first duration and a pause of a second duration, wherein the first duration is either longer or shorter than the second duration.

In some aspects, the sensor comprises an acoustic sensor, and the controller is configured to modulate the at least one stimulation parameter in response to the voice command from the human subject. In some aspects, the controller may be configured to recognize and/or respond to a plurality of voice commands (alone or in addition to tactile input(s)), which may be configured, e.g., to trigger different modulation responses. For example, the controller may be configured to modulate the pulse width of the electrical stimulation pulses based on or in response to a first voice command, and to modulate the pulse frequency of the electrical stimulation pulses based on or in response to a second voice command.

In some aspects, the controller is configured to modulate the at least one stimulation parameter based at least in part on a predefined pattern of tactile input and the voice command from the human subject. In some embodiments, the predefined pattern of tactile input sensed by the motion sensor may include taps and/or bodily gestures.

In some aspects, the system comprises a motion sensor and an acoustic sensor, the predefined pattern of tactile input comprises a double-tap or a triple-tap detected by the motion sensor, and the voice command comprises an audible command provided by the human subject; and the controller is configured to modulate the at least one stimulation parameter based at least in part on the tactile input and the voice command from the human subject, wherein the modulation occurs only if the voice command is validated.

In some aspects, the at least one sensor comprises an acoustic sensor and the controller is configured to a) place the system in a training mode wherein the system is configured to generate, transmit, and/or store a unique identifier associated with a voice of the human subject; and b) validate the voice command using the unique identifier. In some aspects, the validation is performed at least in part using a cloud-based service.

In some aspects, the at least one sensor comprises a motion sensor and the controller is configured to: a) place the system in a training mode wherein the system is configured to obtain training data based on a second predefined pattern of taps and/or one or more gestures, different from a first predefined pattern of taps and/or gesture(s) for modulating at least one of the stimulation parameters (e.g., stimulus amplitude—e.g., current amplitude and/or voltage amplitude; pulse width; duty cycle; and/or frequency), by the human subject, and b) validate the tactile input using the training data, before modulating the at least one stimulation parameter.

In some aspects, the training data comprises timing, location, duration, acceleration, and/or directionality data based on a predefined pattern of taps that includes one or more double-taps and/or triple-taps by the human subject, wherein the predefined pattern of taps is distinct from the predefined pattern of taps for modulating the at least one stimulation parameter.

In some aspects, the sensor comprises a motion sensor and the tactile input comprises a double-tap or a triple-tap detected by the motion sensor; and wherein the controller is configured to modulate the at least one stimulation parameter differently, or to modulate different stimulation parameters, based upon whether a double-tap or a triple-tap is detected. For example, the controller may be configured to increase/decrease the pulse amplitude, pulse width, and/or pulse frequency to a first value (or by a percentage) in response to a double-tap, or to a second value (or by a second percentage) in response to a triple-tap. Alternatively, in some aspects a double-tap may be used as a trigger to boost any of the stimulation parameters described herein, and a triple-tap may be used to cancel the boost, or vice-versa.

In some aspects, the system is further configured to provide audio, visual, and/or haptic feedback: a) in response to the detection of a tactile input and/or a voice command from the human subject; and/or b) before, simultaneously with, or after, the modulation of at least one stimulation parameter of the electrical stimulation pulses.

In some aspects, the audio, visual, and/or haptic feedback is provided by the VNS stimulator or an external device (e.g., a dedicated controller, or software on a computer, tablet, mobile phone, or smartphone paired with or otherwise communicatively linked with the VNS system).

In some aspects, the controller is configured to store one or more profiles comprising a setting for the modulation of the at least one stimulation parameter. In some aspects, the controller may store multiple profiles, e.g., with each profile including settings for the modulation of a plurality of stimulation parameters.

In some aspects, the setting for the modulation of the at least one stimulation parameter comprises a pulse amplitude, pulse width, and/or pulse frequency setting for the electrical stimulation pulses.

In some aspects, the controller is configured to allow the human subject and/or a remote operator (e.g., a programmer clinician) to adjust the setting(s) for the modulation of the at least one stimulation parameter.

In some aspects, the controller is configured to stop and/or revert the modulation of the at least one stimulation parameter based at least in part on a second tactile input (or bodily gesture), and/or a second voice command from the human subject.

In some aspects, the modulation of the at least one stimulation parameter of the electrical stimulation pulses comprises increasing (or decreasing) a pulse amplitude, pulse width, and/or pulse frequency of the stimulation parameter, e.g., based on a percentage of the current value of the stimulation parameter. For example, modulation may comprise increasing/decreasing the stimulation parameter by 1, 2, 3, 4, 5, 6, 7, 8, 9, 10, 15, 20, 25, 30, 35, 40, 45, or 50%, or by a value within a range with endpoints defined by any pair of the foregoing values). In some aspects, the value of the increase (or the decrease) is selected based on one or more preset values, e.g., stored in a profile on the VNS system or available on a communicatively-linked device or server.

In some aspects, the modulation of the at least one stimulation parameter comprises changing the pulse amplitude, pulse width, pulse frequency, ramp-on rate, and/or ramp-off rate of the electrical stimulation pulses. The change may be an increase or a decrease, and the new value(s) may be selected, e.g., using stored preset values, or calculated as a percentage increase or decrease as compared to the current value(s).

In a second general aspect, the disclosure provides methods for VNS using any of the systems described herein. For example, in some aspects, such methods may comprise: a) providing a VNS stimulator implanted in a human subject and configured to transmit electrical stimulation pulses to a vagus nerve of the human subject; b) detecting, by at least one sensor communicatively linked with the VNS stimulator, a tactile input, a bodily gesture, and/or a voice command from the human subject, and c) modulating, by a controller communicatively linked with the at least one sensor and the VNS stimulator, at least one stimulation parameter of the electrical stimulation pulses based at least in part on the detected tactile input, bodily gesture, and/or voice command from the human subject.

In some aspects, the at least one sensor comprises a motion sensor and the tactile input comprises a double-tap or a triple-tap gesture by the human subject.

In some aspects, the at least one sensor comprises an acoustic sensor.

In some aspects, the modulation of the at least one stimulation parameter of the electrical stimulation pulses comprises increasing a pulse amplitude, pulse width, and/or pulse frequency of the stimulation parameter.

In some aspects, the modulation of the at least one stimulation parameter comprises changing the pulse amplitude, pulse width, pulse frequency, ramp-on rate, and/or ramp-off rate of the electrical stimulation pulses. For example, modulation may comprise increasing/decreasing the stimulation parameter by 1, 2, 3, 4, 5, 6, 7, 8, 9, 10, 15, 20, 25, 30, 35, 40, 45, or 50%, or by a value within a range with endpoints defined by any pair of the foregoing values). In some aspects, the value of the increase (or decrease) is selected based on one or more preset values (e.g., stored in a profile on the VNS system or available on a communicatively-linked device or server). In some aspects, the method may comprise modulating a plurality of stimulation parameters in response to a tactile input, bodily gesture, and/or voice command from the human subject

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a VNS system for treating epilepsy using an implantable VNS stimulator coupled via a lead wire to a cuff electrode/cathode on the vagus nerve.

FIG. 2 is a diagram illustrating an exemplary embodiment of a VNS stimulator including a pulse generator and a controller configured to modulate one or more parameters (e.g., a pulse amplitude, pulse width, duty cycle, pulse frequency, ramp-on rate, and/or ramp-off rate) of the electrical stimulation pulses.

FIG. 3 is a diagram illustrating an exemplary embodiment of a wire and electrode cuff for use in VNS stimulation.

FIG. 4 is a conceptual flow diagram of a process for modulating VNS stimulation according to an exemplary embodiment.

FIG. 5 is a conceptual flow diagram of a process for modulating VNS stimulation according to another exemplary embodiment.

FIG. 6 is a graph depicting an increase in stimulation duration for each manual boost (e.g., triggered by tactile input or a voice command) within a 0.5 hour window, along with boost feedback on the synced x-axis.

FIG. 7 is a graph depicting an increase in pulse amplitude for each manual boost (e.g., triggered by tactile input or a voice command) within a 0.5 hour window.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” or “controller” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

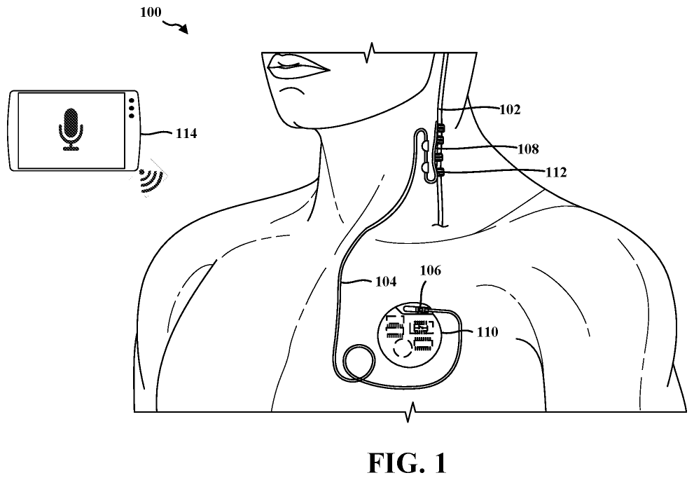

FIG. 1 is a diagram 100 illustrating an exemplary embodiment of a VNS system for treating epilepsy using a VNS stimulator 110 implanted under the skin in the chest of a patient and coupled via a lead wire 104 to a cuff electrode 108 on the vagus nerve 102. In the example shown, four electrodes pairs 112 are used to contact the vagus nerve. The electrodes may be selectively activated, e.g., to identify acceptable amplitudes for the different cathodes. In some embodiments, single electrodes are used. In other embodiments, pairs of electrodes are used. The stimulation of the vagus nerve using electrical pulses is believed to stabilize abnormal electrical activity in the brain which can lead to seizures. VNS stimulator 110 may also include a rechargeable or a primary cell, single-use battery for transmitting electrical pulses using a selectable pulse width and frequency. VNS stimulator 110 may further include an outlet portion for enabling a connection between VNS stimulator 110 and lead wire 104, which enables current flow to the vagus nerve 102 via the cuff 108 and electrode(s) 112.

In some aspects, the VNS system 100 may comprise one or more sensors (e.g., one or more motion sensors or acoustic sensors) configured to detect tactile input, audio signals, voice commands, or other signals from the human subject, a clinician, an operator, or from the environment. These signals may be used to control the VNS system (e.g., to switch between a normal operating mode and a training mode, or to trigger a manual boost in stimulation). For example, the VNS stimulator 110 may include a motion sensor configured to detect one or more taps in proximity to the VNS stimulator 100 (e.g., a single-tap, a double-tap, a triple-tap, or another predefined pattern of taps). Upon detection of a particular tapping pattern (e.g., a double-tap), the VNS system 100 may be configured to modulate one or more stimulation parameters (e.g., by increasing the pulse amplitude, pulse width, duty cycle, pulse frequency, ramp-on rate, and/or ramp-off rate), providing a manual boost in stimulation. This “boost” mode may be applied temporarily, for a predetermined duration, or until the human subject or a clinician or other party triggers cessation or a reversion to previous stimulation parameters (e.g., by providing additional tactile input, such as a triple-tap gesture). It is understood that the VNS system 100 may include a motion sensor as part of or within the VNS stimulator 110, as a separate external or implanted device (e.g., capable of communicating with the VNS stimulator 110 or other components of the VNS system 100 via a wired or wireless connection).

In some aspects, when stimulation is applied by the VNS system 100, there will be a period T₁ of a train of stimulus pulses. Then a period of no stimulation T₂ (referred to as a quiescent period). This cycle may repeat any number of times—i.e., with another period T₁ for a train of stimulus pulses, followed by another quiescent period T₂, continuing until stimulation is terminated by a controller 220 of the VNS system 100. The “duty cycle” of stimulation may be calculated using T₁ and T₂, and expressed as a fraction (duty cycle=T₁/(T₁+T₂)) or alternatively as a percentage (duty cycle=(100×T₁)/(T₁+T₂)). Thus, references to the term “duty cycle” herein should be understood as references to a parameter as defined by the foregoing calculations.

As noted above, the VNS system 100 may include one or more acoustic sensors configured to detect audible input (e.g., voice commands). FIG. 1 illustrates an exemplary embodiment wherein an acoustic sensor is incorporated into an electronic device 114 configured to control the VNS system 100. Device 114 may be a smart phone, a smart watch or a dedicated programmer. The device 114 can include acoustic sensors which can detect voice commands. In some embodiments, an acoustic sensor which can detect voice commands can be contained in the VNS Stimulator 110. In some aspects, the electronic device may be a computer, tablet, or other general purpose computing platform capable of communicating with the VNS stimulator 110 or other components of the VNS system 100 via a wired or wireless connection. In some aspects, the electronic device may be a dedicated controller device (e.g., provided with the VNS system 100). The VNS system 100 may be configured to allow the human subject to control one or more stimulation parameters using voice commands, alone or in combination with tactile input. For example, the VNS system 100 may be configured to modulate the pulse amplitude, pulse width, pulse frequency, ramp-on rate, and/or ramp-off rate based on a voice command programmed as a trigger for a “boost” mode, wherein the amount, intensity, duration, and/or level of one or more of these parameters may be increased for a predetermined duration, or until the human subject or a clinician or other party triggers cessation or a reversion to previous stimulation parameters (e.g., the VNS system 100 may be programmed to end the “boost” mode and to return to a previous set of stimulation parameters in response to a second voice command from the human subject, clinician or other party).

In some aspects, the VNS system 100 may be configured to utilize both tactile input and voice commands (or other audible signals). For example, an exemplary VNS system 100 may include both a motion sensor (e.g., included in the housing of the VNS stimulator 110) and an acoustic sensor (e.g., in an electronic device communicatively linked with the VNS system 100). Such systems may be advantageous in some use cases because they can reduce the possibility that a modulation of stimulation parameters is triggered inadvertently (e.g., due to inadvertent physical contact in proximity to the motion sensor being detected as a double- or triple-tap gesture intended to trigger modulation). For example, a VNS system 100 may be configured to require vocal input from the human subject in order to confirm that modulation is in fact desired after the detection of a predefined pattern of tactile input by the motion detector. In some exemplary aspects, voice commands may be similarly paired with bodily gestures (e.g., a voice command may be required to confirm modulation triggered by a bodily gesture).

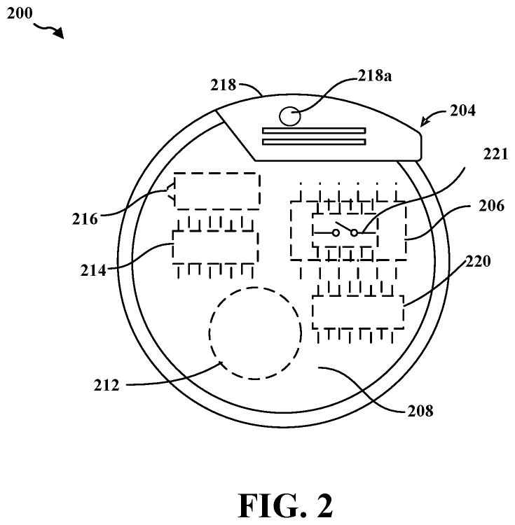

FIG. 2 is a diagram 200 illustrating an exemplary embodiment of a VNS stimulator 204 including a controller 220 with processing circuitry configured to modulate at least one stimulation parameter of the electrical stimulation pulses based at least in part on the detection of a predefined pattern of tactile input, a bodily gesture, and/or a voice command from the human subject, as described in greater detail herein. The VNS stimulator 204 may include a motion sensor 212 (e.g. a one- or two-axis accelerometer). The VNS stimulator 204 may further comprise a pulse generator 206 which is programmed to generate a periodic electrical pulse having a set frequency and pulse width. The pulse generator may be battery-powered and can be activated and deactivated (the latter causing the current to be turned off) via a switch 221. The connections on the integrated circuits may be coupled together selectively via a small printed circuit board 208. In other embodiments, the VNS stimulator 204 may be implemented as an SoC on a die, or a packaged die.

VNS stimulator 204 further may include a transceiver/receiver 216. In some embodiments, transceiver 216 includes a wireless receiver configured to receive wireless signals, e.g., Bluetooth Low Energy, from a source external to the patient (e.g., for communication with an external or implanted electronic device that incorporates an acoustic sensor capable of receiving voice commands). In some embodiments, the transceiver may further comprise a wireless transmitter, e.g., for providing feedback to a processor used in a clinician programmer device, or to an external sensor. In still other embodiments, the transceiver 216 may include a wire for receiving information from the vagus nerve or another part of the body. The wire may also extend outside the body for temporary connection to an external sensor or other processing device. The wireless receiver in transceiver 216 may further be configured in some embodiments to receive instructions for the controller 220 to modulate one or more parameters of the electrical stimulation pulses (e.g., to provide a manual stimulation boost, as described herein). The wireless transceiver 216 may further be configured to receive information including events recorded or detected by one or more external sensors. When acting as a transmitter, the transceiver 216 can provide feedback signals to external sources using data generated by controller 220.

In some embodiments, the information received from the wireless receiver/transceiver 216 may be provided to a memory 214. The controller 220 may access the memory 214 to receive and process instructions to modulate (e.g., increase or decrease the pulse amplitude, pulse width, and/or pulse frequency of) one or more parameters of the generated electrical stimulation pulses, or to temporarily deactivate the pulse generator 206. In various embodiments, the controller 220 may access information including physical events detected by one or more implanted or external sensors (e.g., by a motion sensor or acoustic sensor as described herein). The processor may evaluate the detected events and, in these embodiments, modulate the pulse amplitude, pulse width, pulse frequency, ramp-on rate, and/or ramp-off rate of the electrical stimulation pulses. The pulse generator 206 may generate the electrical stimulation pulses accordingly, which may be provided to an outlet portion 218 to which the lead wire (see FIG. 1) is attached. In one embodiment, the outlet portion may include an aperture 218a for a jack to be inserted to attach the lead wire

In some aspects, the VNS system 100 may be configured to modulate one or more stimulation parameters according to predetermined settings. For example, the VNS system 100 may be configured to adjust the pulse width and amplitude to one level in response to a double-tap, versus another level in response to a triple-tap (or based on any predefined pattern of tactile input or a voice command). In some aspects, a double-tap is particularly useful, because it is not as likely to be inadvertently triggered as a single-tap. However, the VNS stimulator 110 can be configured to recognize a double-tap and assert a discrete output when the double-tap has been detected. Once the double-tap has been detected (e.g., by an accelerometer incorporated into the VNS stimulator 110 as a motion sensor) and reported to the controller 220 of the VNS stimulator 110, stimulation may be delivered using modified stimulation parameters, e.g., for a preset duration or until manually adjusted via a subsequent tactile input or voice command.

In some aspects, the controller 220 may be configured to place the VNS system 100 in a training mode, e.g., to allow the human subject to gain experience with the process of providing tactile input without triggering modulation of stimulation parameters. For example, in training mode the clinician may instruct the human subject to tap on their chest over the implant. In some embodiments, the controller 220 may be configured to provide feedback to the human subject during training mode (e.g., when the human subject taps on their chest over the implant, audio, visual, or haptic feedback could emanate from the implant or from an external device). In some aspects, the feedback may comprise audio or visual feedback from an dedicated controller, or from an application executed on a paired device such as a computer, tablet or other electronic device. This feedback could indicate if the tap was adequate to cause a therapeutic effect when the implant is not in training mode.

In some aspects, a training mode may also be used to train the implant. For example, the timing between each of the taps provided by a human subject could be recorded during training. Then, upon completion of the training, the controller 220 may be configured to use a timing window (e.g., minimum and maximum times) based on the training data, when determining if a series of two taps should be treated as a double-tap. In this training mode, the VNS system 100 may also be configured to determine the direction of the acceleration resulting from a tap. This data may be used by the controller 220 to make the tap interpretation only responsive to taps from a specific direction, e.g., to detect and respond to taps on the chest of the human subject, rather than on the back.

As indicated above, the VNS system 100 may also be controlled using voice commands or other audible signals (e.g., detected by a microphone or other acoustic sensor). The controller 220 may be configured to respond to voice commands instead of or in addition to tactile input in order to initiate a therapeutic boost or to otherwise modulate one or more stimulation parameters. In some aspects, voice commands may be interpreted using a cloud-based voice recognition service provider such as Amazon Web Services (AWS) or edge-based voice services (e.g., software executed by the VNS system 100 or a local electronic device communicatively linked to the VNS system 100. In some aspects, the VNS system 100 may provide a voice training mode, configured to obtain one or more samples of the human subject's voice in order to generate a unique identifier associated with the human subject. Such systems may advantageously be used to avoid false-positive or otherwise inadvertent voice commands. In some aspect, the VNS system 100 may be configured to utilize tactile input and voice commands, e.g., as a safety feature to avoid inadvertent modulation of stimulation parameters. For example, the controller 220 may be configured to require a particular voice command as confirmation following the detection of tactile input set as a trigger for modulation of stimulation parameters.

Sometimes patients will sense multiple impending seizures and respond to them prior to an actual seizure occurring. Thus, in some aspects it would be beneficial for the VNS system 100 to keep track of the number of manual stimulation boost requests over a fixed-duration time window and increase one or more of the stimulation parameters more than with a single manual boost request. For example, the controller 220 may be configured to count the number of tactile input and/or voice command requests for a boost as to one or more stimulation parameters during a window of time (e.g., 10, 20, 30, 40, 50, 60, 70, 80, or 90 seconds) and to provide a greater increase in the pulse amplitude, pulse width, and/or pulse frequency of one or more stimulation parameters when a threshold number of boost requests is detected during the window of time. Alternatively, in some aspects, multiple manual boost requests during a time window may be used as a trigger to increase the stimulation boost duration. For example, a single double-tap could result in a 0.2 mA boost with a 30 s duration, whereas if a second double-tap is detected within 30 minutes of the first, the VNS system 100 might deliver a 0.2 mA boost with a 60 s duration. In some aspects, the increase in duration may be extended further in the following manner: if n is the number of boost requests in the last 30 minutes, the implant might deliver stimulation at 0.2 mA above the background stimulation amplitude for n*30 seconds. In other aspects, other algorithms may be used to determine the increase in stimulation duration.

In some aspect, the VNS system 100 may be configured to provide feedback to the human subject as a direct indication that system is working on a request. The feedback may be provided, e.g., in the form of vibration using a haptic device and/or an audio notification generated by the implanted VNS stimulator 110 or by a connected external instrument (a dedicated controller, software executed on a communicatively-linked computer or tablet, phone, watch etc.). By providing feedback, the VNS system 100 may eliminate cases of accidental additional boost(s) as the human subject may receive feedback as soon as boost is being applied upon detection of a double-tap or other tactile input (or voice command) by the VNS system 100. In some aspects, the feedback may be vibration for up to 1, 2, 3, 4, 5, 6, 7, 8, 9 or 10 seconds and/or a notification on an application or watch within first 1, 2, 3, 4, 5, 6, 7, 8, 9, 10, 20, or 30 seconds, etc., of manual boost stimulation.

While various functions of the VNS stimulator 204 have been shown, it will be appreciated by those skilled in the art upon review of this disclosure that different architectures may be used. For example, the controller 220 may include more than one integrated circuit, or it may include a separate module coupled to the VNS stimulator. The controller 220 may further include one or more general purpose processors, RISC processors, or other types of processors. The controller 220 may in some embodiments include dedicated hardware. For example, the controller 220 may be any one or more of a digital signal processor (DSP), a field programmable gate array (FPGA), an application-specific integrated circuit (ASIC), or a combination of digital logic devices.

The memory 214 may include any suitable memory, such as a combination of volatile and non-volatile memory, dynamic random access memory (DRAM), static random access memory (SRAM), read only memory, flash or other solid state memory, or the like. Other types of memory are possible. Non-volatile memory within memory 214 may be used to store critical settings to enable system reset, for example. Pulse frequency, pulse amplitude, and/or pulse width values may be stored in the memory. In some embodiments, the memory may be accessible and programmable as noted above via the controller 220, or via data or instructions received at via wireless receiver 216. The memory 214 may also include firmware for use by the controller, or other program information for automatically modulating one or more parameters of the electrical pulses in response to a tactile input or voice command by the human subject, a clinician, or another authorized operator. In some embodiments, the modulation of stimulation parameters may be based, in part or in whole, on information received at the receiver 216 such as detected events (e.g., a voice command detected by the acoustic sensor incorporated into an external electronic device sensors). It will be appreciated that VNS stimulator 204 need not be circular or elliptical in nature, and may take on different shapes based on different design considerations and patient needs. More generally, the components identified in the various figures may take on different geometries than those shown.

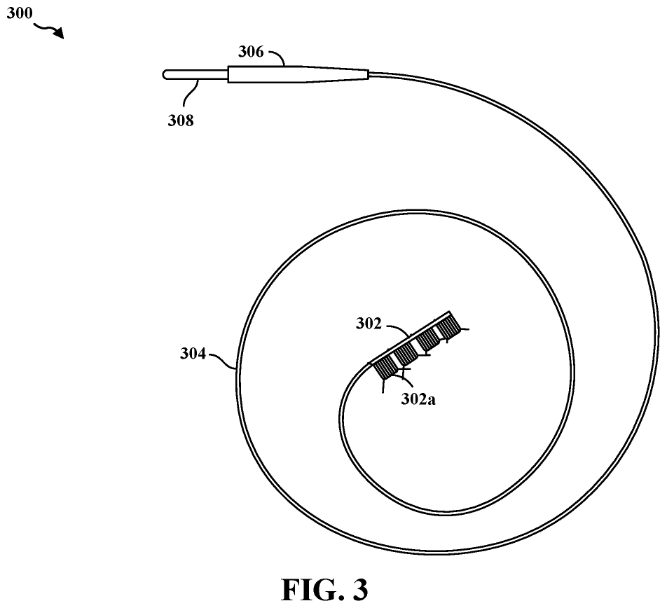

FIG. 3 is a diagram illustrating an exemplary embodiment of a wire and electrode cuff for use in VNS stimulation. The jack 308 may be inserted in one configuration into the aperture 218a (FIG. 2) of VNS stimulator 204. It will be appreciated that the components are not drawn to scale, and typically the jack 308 in this embodiment would be sized very small, and/or angled differently, to be minimally intrusive to the patient in which the component is implanted. In some embodiments, an insulating sheath 306 may provide support for the jack 308 as the insulated wire 304 terminates at electrode cuff or lead 302, which further illustrates a plurality of electrodes 302a or electrode pairs.

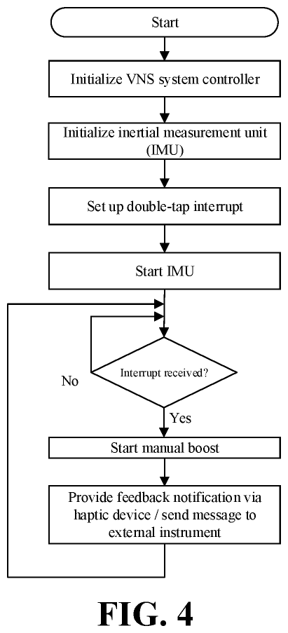

FIG. 4 is a diagram illustrating an exemplary method for modulating VNS using a VNS system 100 according to the disclosure. In this example, the controller 210 and an inertial measurement unit (“IMU”; e.g., an accelerometer operating as a motion sensor) are first initialized. An interrupt is then set up (e.g., in this case to detect the occurrence of a double-tap gesture). The IMU is then activated in order to obtain motion data. Upon detection of a double-tap, the interrupt is triggered resulting in activation of a manual boost (e.g., upwards modulation of one or more stimulation parameters). After activating the boost, feedback is provided to the human subject (e.g., haptic feedback or a message prompt). The controller may then return to monitoring to the IMU data stream in order to determine whether an additional double-tap (or other tactile input) has been received.

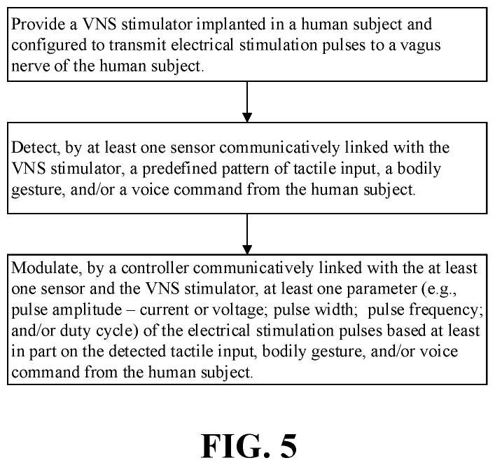

FIG. 5 is a diagram illustrating another exemplary method for modulating VNS using a VNS system 100 according to the disclosure. As illustrated by this diagram, such methods may be performed using a VNS stimulator implanted in a human subject and configured to transmit electrical stimulation pulses to a vagus nerve of the human subject. This system may be used to detect by at least one sensor communicatively linked with the VNS stimulator, a predefined pattern of tactile input and/or a voice command from the human subject. In response, a controller communicatively linked with the at least one sensor and the VNS stimulator, may modulate at least one parameter of the electrical stimulation pulses based at least in part on the detected tactile input and/or the voice command from the human subject. In some aspects, modulation may entail an increase (or boost) in the pulse amplitude, pulse width, and/or pulse frequency of the electrical stimulation pulses.

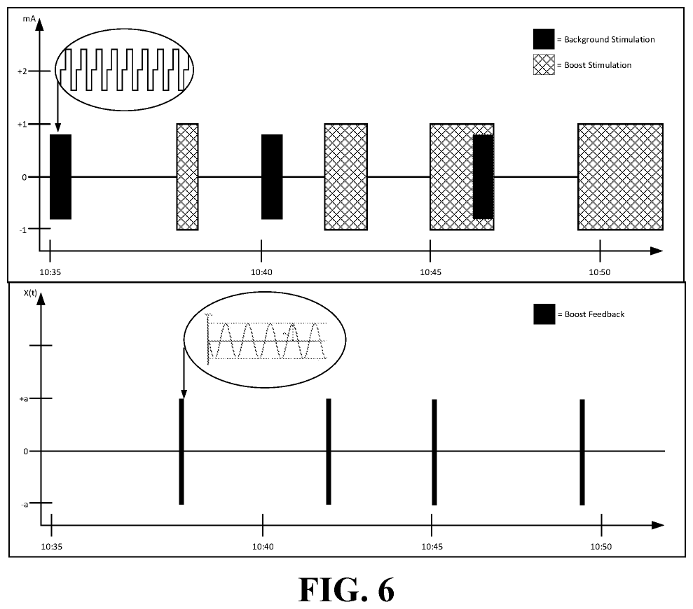

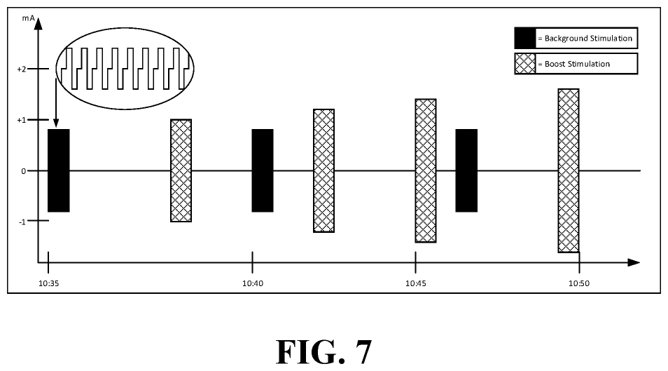

FIG. 6 illustrates an increase in the stimulation boost duration for each manual boost within a 0.5 hour window, along with boost feedback on the synced x-axis. Similarly, FIG. 7 illustrates an increased pulse amplitude for each manual boost within a 0.5 hour window. As shown by these figures, multiple boosts may be requested as-needed by a human subject (e.g., in response to the belief that a seizure is imminent).

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

### EXEMPLARY EMBODIMENTS

The following set of exemplary embodiments is non-limiting and provided merely to illustrate aspects of the present disclosure. It should be understood that additional embodiments may comprise any other elements described herein, alone or in combination, and may also provide any additional functional aspects described herein.

Embodiment 1. A system for vagus nerve stimulation (VNS), comprising:

a VNS stimulator implanted in a human subject and configured to transmit electrical stimulation pulses to a vagus nerve of the human subject; and

at least one sensor configured to detect a predefined pattern of tactile input, a bodily gesture, and/or a voice command from the human subject,

wherein the VNS stimulator comprises a controller configured to modulate at least one stimulation parameter of the electrical stimulation pulses based at least in part on the tactile input, bodily gesture, and/or voice command from the human subject.

Embodiment 2. The system for VNS of Embodiment 1, wherein the at least one stimulation parameter comprises a pulse amplitude, pulse width, duty cycle, pulse frequency, ramp-on rate, and/or ramp-off rate of the electrical stimulation pulses.

Embodiment 3. The system for VNS of Embodiments 1 or 2, wherein the controller is configured to modulate the at least one stimulation parameter in response to the predefined pattern of tactile input from the human subject, wherein the tactile input comprises one or more taps that are detected by the sensor.

Embodiment 4. The system for VNS of Embodiments 2 or 3, wherein the sensor comprises a motion sensor and the predefined pattern of tactile input comprises a double-tap detected by the motion sensor.

Embodiment 5. The system for VNS of any one of Embodiments 2-4, wherein the sensor comprises a motion sensor and the predefined pattern of tactile input comprises a triple-tap detected by the motion sensor.

Embodiment 6. The system for VNS of any one of Embodiments 2-5, wherein the sensor comprises an acoustic sensor, and the controller is configured to modulate the at least one stimulation parameter in response to the voice command from the human subject, wherein the acoustic sensor is located in or on the VNS stimulator or is located on an external device.

Embodiment 7. The system for VNS of any one of Embodiments 2-6, wherein the controller is configured to modulate the at least one stimulation parameter based at least in part on the predefined pattern of tactile input and the voice command from the human subject.

Embodiment 8. The system for VNS of Embodiment 7, wherein

wherein the system comprises a motion sensor and an acoustic sensor,

the predefined pattern of tactile input comprises two or more taps detected by the motion sensor, and

the voice command comprises an audible command provided by the human subject; and

wherein the controller is configured to modulate the at least one stimulation parameter based at least in part on the predefined pattern of tactile input or the voice command from the human subject, wherein in the case of a voice command the modulation occurs only if the voice command has been validated.

Embodiment 9. The system for VNS of any one of Embodiments 2-8, wherein the at least one sensor comprises an acoustic sensor and the controller is configured to

a) place the system in a training mode wherein the system is configured to generate, transmit, and/or store a unique identifier associated with a voice of the human subject; and

b) validate the voice command using the unique identifier.

Embodiment 10. The system for VNS of Embodiment 9, wherein the validation is performed at least in part using a cloud-based service.

Embodiment 11. The system for VNS of any one of Embodiments 2-10, wherein the at least one sensor comprises a motion sensor and the controller is configured to:

a) place the system in a training mode wherein the system is configured to obtain training data based on a second predefined pattern of tactile input and/or bodily gesture, different from a first predefined pattern of tactile input and/or bodily gesture, for modulating the at least one stimulation parameter, optionally wherein the at least one stimulation parameter comprises an amplitude, pulse width, duty cycle, and/or pulse frequency of the electrical stimulation pulses, by the human subject, and

b) validate the tactile input and/or bodily gesture using the training data, before modulating the at least one stimulation parameter.

Embodiment 12. The system for VNS of Embodiment 11, wherein the training data comprises timing, location, duration, acceleration, and/or directionality data based on a predefined pattern of taps that includes two or more taps by the human subject, wherein the predefined pattern of taps is distinct from the predefined pattern of taps for modulating the at least one stimulation parameter.

Embodiment 13. The system for VNS of any one of Embodiments 2-12, wherein the sensor comprises a motion sensor and the tactile input comprises a double-tap or a triple-tap detected by the motion sensor; and wherein the controller is configured to modulate the at least one stimulation parameter differently based upon whether a double-tap or a triple-tap is detected.

Embodiment 14. The system for VNS of any one of Embodiments 2-13, wherein the system is further configured to provide audio, visual, and/or haptic feedback:

a) in response to the detection of the tactile input and/or the voice command from the human subject; and/or

b) before, simultaneously with, or after, the modulation of the at least one stimulation parameter of the electrical stimulation pulses;

Embodiment 15. The system for VNS of Embodiment 14, wherein the audio, visual, and/or haptic feedback is provided by the VNS stimulator or an external device, optionally wherein the external device is a dedicated controller or a computer, tablet, or phone that has been communicatively-linked with the VNS stimulator.

Embodiment 16. The system of any one of Embodiments 1-15, wherein the controller is configured to store one or more profiles comprising a setting for the modulation of the at least one stimulation parameter.

Embodiment 17. The system of Embodiment 16, wherein the setting for the modulation of the at least one stimulation parameter comprises a pulse amplitude, pulse width, and/or pulse frequency setting for the stimulation parameter.

Embodiment 18. The system of Embodiment 16, wherein the controller is configured to allow the human subject and/or a remote operator to adjust the setting for the modulation of the at least one stimulation parameter.

Embodiment 19. The system of any one of Embodiments 1-18, wherein the controller is configured to stop and/or revert the modulation of the at least one stimulation parameter based at least in part on a second tactile input and/or second voice command from the human subject.

Embodiment 20. The system of any one of Embodiments 1-19, wherein the modulation of the at least one stimulation parameter comprises increasing the pulse amplitude, pulse width, and/or pulse frequency of the electrical stimulation pulses.

Embodiment 21. The system of any one of Embodiments 1-20, wherein the modulation of the at least one stimulation parameter comprises changing the pulse amplitude, pulse width, duty cycle, pulse frequency, ramp-on rate, and/or ramp-off rate of the electrical stimulation pulses, optionally wherein

a) a new setting for the stimulation parameter is selected as a percentage increase or decrease in value based on a current value for the stimulation parameter being modulated; or

b) a new setting for the stimulation parameter is selected from one or more preset values stored in a memory component of the VNS stimulator or a dedicated controller, computer, tablet, smartwatch, mobile phone or smartphone that has been communicatively linked with the VNS stimulator.

Embodiment 22. A method for vagus nerve stimulation (VNS), comprising:

a) providing a VNS stimulator implanted in a human subject and configured to transmit electrical stimulation pulses to a vagus nerve of the human subject;

b) detecting, by at least one sensor communicatively linked with the VNS stimulator, a predefined pattern of tactile input, a bodily gesture, and/or a voice command from the human subject, and

c) modulating, by a controller communicatively linked with the at least one sensor and the VNS stimulator, at least one parameter of the electrical stimulation pulses based at least in part on the detected tactile input, bodily gesture, and/or voice command from the human subject.

Embodiment 23. The method of Embodiment 22, wherein the at least one sensor comprises a motion sensor and the tactile input comprises a double-tap or a triple-tap by the human subject.

Embodiment 24. The method of Embodiments 22 or 23, wherein the at least one sensor comprises an acoustic sensor.

Embodiment 25. The method of any one of Embodiments 22-24, wherein the modulation of the at least one parameter of the electrical stimulation pulses comprises increasing or decreasing a pulse amplitude, pulse width, duty cycle, pulse frequency, ramp-on rate, and/or ramp-off rate.

Embodiment 26. The method of any one of Embodiments 22-25, wherein the modulation of the at least one parameter of the electrical stimulation pulses comprises changing the pulse amplitude, pulse width, duty cycle, pulse frequency, ramp-on rate, and/or ramp-off rate, optionally wherein

a) a new setting for the parameter is selected as a percentage increase or decrease in value based on a current value for the parameter being modulated; or

b) a new setting for the parameter is selected from one or more preset values stored in a memory component of the VNS stimulator or a dedicated controller, computer, tablet, smartwatch, mobile phone, or smartphone that has been communicatively linked with the VNS stimulator.

What is claimed is:

1. A system for vagus nerve stimulation (VNS), comprising:
a VNS stimulator implanted in a human subject and configured to transmit electrical stimulation pulses to a vagus nerve of the human subject; and
at least one sensor configured to detect a predefined pattern of tactile input, a bodily gesture, and/or a voice command from the human subject,
wherein the VNS stimulator comprises a controller configured to modulate at least one stimulation parameter of the electrical stimulation pulses based at least in part on the tactile input, bodily gesture, and/or voice command from the human subject.

2. The system for VNS of claim 1, wherein the at least one stimulation parameter comprises a pulse amplitude, pulse width, duty cycle, pulse frequency, ramp-on rate, and/or ramp-off rate of the electrical stimulation pulses.

3. The system for VNS of claim 2, wherein the controller is configured to modulate the at least one stimulation parameter in response to the predefined pattern of tactile input from the human subject, wherein the tactile input comprises one or more taps that are detected by the sensor.

4. The system for VNS of claim 2, wherein the sensor comprises a motion sensor and the predefined pattern of tactile input comprises a double-tap detected by the motion sensor.

5. The system for VNS of claim 2, wherein the sensor comprises a motion sensor and the predefined pattern of tactile input comprises a triple-tap detected by the motion sensor.

6. The system for VNS of claim 2, wherein the sensor comprises an acoustic sensor, and the controller is configured to modulate the at least one stimulation parameter in response to the voice command from the human subject, wherein the acoustic sensor is located in or on the VNS stimulator or is located on an external device.

7. The system for VNS of claim 2, wherein the controller is configured to modulate the at least one stimulation parameter based at least in part on the predefined pattern of tactile input and the voice command from the human subject.

8. The system for VNS of claim 7, wherein
wherein the system comprises a motion sensor and an acoustic sensor,
the predefined pattern of tactile input comprises two or more taps detected by the motion sensor, and
the voice command comprises an audible command provided by the human subject; and
wherein the controller is configured to modulate the at least one stimulation parameter based at least in part on the predefined pattern of tactile input or the voice command from the human subject, wherein in the case of a voice command the modulation occurs only if the voice command has been validated.

9. The system for VNS of claim 2, wherein the at least one sensor comprises an acoustic sensor and the controller is configured to
a) place the system in a training mode wherein the system is configured to generate, transmit, and/or store a unique identifier associated with a voice of the human subject; and
b) validate the voice command using the unique identifier.

10. The system for VNS of claim 9, wherein the validation is performed at least in part using a cloud-based service.

11. The system for VNS of claim 2, wherein the at least one sensor comprises a motion sensor and the controller is configured to:
a) place the system in a training mode wherein the system is configured to obtain training data based on a second predefined pattern of tactile input and/or bodily gesture, different from a first predefined pattern of tactile input and/or bodily gesture, for modulating the at least one stimulation parameter, optionally wherein the at least one stimulation parameter comprises an amplitude, pulse width, duty cycle, and/or pulse frequency of the electrical stimulation pulses, by the human subject, and
b) validate the tactile input and/or bodily gesture using the training data, before modulating the at least one stimulation parameter.

12. The system for VNS of claim 11, wherein the training data comprises timing, location, duration, acceleration, and/or directionality data based on a predefined pattern of taps that includes two or more taps by the human subject, wherein the predefined pattern of taps is distinct from the predefined pattern of taps for modulating the at least one stimulation parameter.

13. The system for VNS of claim 2, wherein the sensor comprises a motion sensor and the tactile input comprises a double-tap or a triple-tap detected by the motion sensor; and
wherein the controller is configured to modulate the at least one stimulation parameter differently based upon whether a double-tap or a triple-tap is detected.

14. The system for VNS of claim 2, wherein the system is further configured to provide audio, visual, and/or haptic feedback:
a) in response to the detection of the tactile input and/or the voice command from the human subject; and/or
b) before, simultaneously with, or after, the modulation of the at least one stimulation parameter of the electrical stimulation pulses.

15. The system for VNS of claim 14, wherein the audio, visual, and/or haptic feedback is provided by the VNS stimulator or an external device, optionally wherein the external device is a dedicated controller or a computer, tablet, or phone that has been communicatively-linked with the VNS stimulator.

16. The system of claim 1, wherein the controller is configured to store one or more profiles comprising a setting for the modulation of the at least one stimulation parameter.

17. The system of claim 16, wherein the setting for the modulation of the at least one stimulation parameter comprises a pulse amplitude, pulse width, and/or pulse frequency setting for the stimulation parameter.

18. The system of claim 16, wherein the controller is configured to allow the human subject and/or a remote operator to adjust the setting for the modulation of the at least one stimulation parameter.

19. The system of claim 1, wherein the controller is configured to stop and/or revert the modulation of the at least one stimulation parameter based at least in part on a second tactile input and/or second voice command from the human subject.

20. The system of claim 1, wherein the modulation of the at least one stimulation parameter comprises increasing the pulse amplitude, pulse width, and/or pulse frequency of the electrical stimulation pulses.

21. The system of claim 1, wherein the modulation of the at least one stimulation parameter comprises changing the pulse amplitude, pulse width, duty cycle, pulse frequency, ramp-on rate, and/or ramp-off rate of the electrical stimulation pulses, optionally wherein
a) a new setting for the stimulation parameter is selected as a percentage increase or decrease in value based on a current value for the stimulation parameter being modulated; or
b) a new setting for the stimulation parameter is selected from one or more preset values stored in a memory component of the VNS stimulator or a dedicated controller, computer, tablet, smartwatch, mobile phone or smartphone that has been communicatively linked with the VNS stimulator.

22. A method for vagus nerve stimulation (VNS), comprising:
a) providing a VNS stimulator implanted in a human subject and configured to transmit electrical stimulation pulses to a vagus nerve of the human subject;
b) detecting, by at least one sensor communicatively linked with the VNS stimulator, a predefined pattern of tactile input, a bodily gesture, and/or a voice command from the human subject, and
c) modulating, by a controller communicatively linked with the at least one sensor and the VNS stimulator, at least one parameter of the electrical stimulation pulses based at least in part on the detected tactile input, bodily gesture, and/or voice command from the human subject.

23. The method of claim 22, wherein the at least one sensor comprises a motion sensor and the tactile input comprises a double-tap or a triple-tap by the human subject.

24. The method of claim 22, wherein the at least one sensor comprises an acoustic sensor.

25. The method of claim 22, wherein the modulation of the at least one parameter of the electrical stimulation pulses comprises increasing or decreasing a pulse amplitude, pulse width, duty cycle, pulse frequency, ramp-on rate, and/or ramp-off rate.

26. The method of claim 22, wherein the modulation of the at least one parameter of the electrical stimulation pulses comprises changing the pulse amplitude, pulse width, duty cycle, pulse frequency, ramp-on rate, and/or ramp-off rate, optionally wherein
a) a new setting for the parameter is selected as a percentage increase or decrease in value based on a current value for the parameter being modulated; or
b) a new setting for the parameter is selected from one or more preset values stored in a memory component of the VNS stimulator or a dedicated controller, computer, tablet, smartwatch, mobile phone, or smartphone that has been communicatively linked with the VNS stimulator.
